(19) United States
(12) Patent Application Publication
(10) Pub. No.: US 2024/0065825 A1
(43) Pub. Date: Feb. 29, 2024

(54) DEVICES AND SYSTEMS FOR THE STABILIZATION OF BONES, TISSUES, AND GRAFTS

(71) Applicant: Anza Innovations Inc., Del Mar, CA (US)

(72) Inventor: Eugene Shostaev, Del Mar, CA (US)

(21) Appl. No.: 18/458,925

(22) Filed: Aug. 30, 2023

Related U.S. Application Data
(60) Provisional application No. 63/402,274, filed on Aug. 30, 2022.

(51) Int. Cl. A61F 2/08

(52) U.S. Cl. CPC A61F 2/0811; A61F 2002/0835; A61F 2002/0852

(57) ABSTRACT

Devices and systems for the stabilization of bones, tissues, and grafts can include a knotless, adjustable, self-locking button/loop assembly for orthopedic repair surgeries. A preferred embodiment of the present invention can include an adjustable, knotless button/loop assembly formed of a pair of fixation devices, for example proximal and distal buttons or button assemblies, connected by a knotless, adjustable flexible loop or loops.

### RELATED APPLICATIONS

This application claims the benefit of co-pending U.S. Provisional Application Ser. No. 63/402,274, entitled DEVICES AND SYSTEMS FOR THE STABILIZATION OF BONES, TISSUES, AND GRAFTS, filed Aug. 30, 2022, the entire disclosures of which is herein incorporated by reference.

### FIELD OF THE INVENTION

This invention relates generally to the field of orthopedic surgery and more specifically to ankle, knee, and shoulder surgery, including ankle syndesmosis repair techniques and associated fixation and reconstruction methods and devices.

### BACKGROUND OF THE INVENTION

Ankle injuries are some of the most common of the bone and joint injuries. The ankle joint comprises three bones: the tibia, which makes up the medial aspect; the fibula, which parallels the tibia and makes up the lateral aspect; and the talus. The far ends of the tibia and fibula are called the malleoli and together they comprise an arch sitting on top of the talus. The joint capsule encases the joint and is lined with a smooth layer called the synovium. Synovium produces synovial fluid, which allows for smooth movement of the joint surfaces. The ankle joint is kept stable by three groups of ligaments.

Surgery to fix an ankle fracture is indicated for patients who suffer a displaced ankle fracture involving the tibia, fibula or both. One of the injuries that may occur due to such fracture is disruption of the syndesmosis. A syndesmosis injury is a disruption of the ligaments that hold the fibula and tibia together near the ankle joint. If the syndesmosis is damaged, ripped, or torn, the ankle joint may become unstable and surgery may be indicated. Suture-button constructs for ankle syndesmosis repair have been effective in ankle syndesmosis repair as compared to the prior art, but they frequently require the surgeon to tie knots in order to secure the proximal (lateral) button against the lateral surface of the fibula, or else are unable to adequately maintain the desired bone alignment after the repair. It is desirable to have an ankle syndesmosis repair system which allows for durable syndesmosis repair and fixation without the need for tying knots and that would maintain fixation and resist loosening or lengthening after the repair.

### SUMMARY OF THE INVENTION

In view of the foregoing disadvantages inherent in the known types of devices and systems for stabilizing bones, tissues, and grafts or the like in the prior art, the present stabilization system overcomes disadvantages of the prior art by providing novel devices and systems for stabilizing bones, tissues, and grafts. As such, the present stabilization system provides devices and reconstruction systems and methods for the stabilization of bones, tissues, and grafts including a knotless, adjustable, self-locking button/loop system for ankle syndesmosis and other orthopedic repair surgeries with or without concomitant ankle fracture repair. The stabilization system can include an adjustable, knotless button/loop system formed of a pair of fixation devices (e.g. proximal and distal buttons or button assemblies) connected by a knotless, adjustable flexible loop or loops, with all the advantages of the prior art and none of the disadvantages.

There has thus been outlined, rather broadly, the more important features of the stabilization system in order that the detailed description thereof that follows may be better understood and in order that the present contribution to the art may be better appreciated. These and other features, aspects, and advantages of the present stabilization system will become better understood with reference to the following drawings and

### DETAILED DESCRIPTION

In an embodiment, a fixation system for knotlessly securing a flexible member stabilizing an anatomical structure can include a clamp with a first branch and a second branch that can be adapted to move towards each other to close the clamp, one or more saddles at a distal end of the first branch and the second branch, the saddles adapted for a flexible member to be wrapped around the one or more saddles to form two or more loops around the one or more saddles and an anatomical structure of a patient, a first protuberance at a proximal end of the first branch and a second protuberance at a proximal end of the second branch, and a first jaw on the first protuberance and a second jaw on the second protuberance, the first and second jaws defining a clamping area, the first and second jaws adapted for clamping and knotlessly securing the flexible member within the clamping area between the jaws.

The clamp can include a force receiving area on each of the protuberances, the force receiving areas laterally distant from a distal-proximal central axis of the clamp, the force receiving areas adapted to receive contact force, thereby urging the first branch and the second branch towards each other. The fixation system can include a proximal base having a pocket configured to accommodate the protuberances, the pocket having an opening to allow the one or more saddles and the branches to extend through the opening in a distal direction. The proximal base can include a sloped mating ramp on an interior of the pocket, and the sloped mating ramp can be configured to engage with the force receiving areas and apply contact force to the force receiving areas, thereby urging the first branch and the second branch towards each other, when a force is applied to the one or more saddles in a distal direction. The fixation system can include an expanded footprint washer, and the expanded footprint washer can have a washer recess configured to accommodate the proximal base, and the washer recess can have an opening to allow the one or more saddles and the branches to extend through the opening in a distal direction, and the washer pocket can have a distal surface adapted for contact with an anatomical structure. the force receiving areas can be a distalmost portion of a distal surface of the protuberances. The one or more saddles can a single saddle, and the first branch and the second branch can extend proximally from the saddle, the first branch and the second branch flexing slightly towards each other at their proximal ends to bring the jaws together when a force is applied to the saddle in a distal direction. The one or more saddles can be a first saddle and a second saddle, with the first saddle on a proximal portion of the first branch and the second saddle on a proximal portion of the second branch, with the first saddle and the second saddle configured to be arranged laterally distant from each other so that the flexible member can be looped through each saddle separately, and the first protuberance and the second protuberance can be urged towards each other when a force is applied to the first saddle and the second saddle in a distal direction. The one or more saddles can be a first saddle and a second saddle, with first saddle on a proximal portion of the first branch and the second saddle on a proximal portion of the second branch, with first saddle and the second saddle configured to be stacked together so that the flexible member can be looped through both saddles together, and the first protuberance and the second protuberance can be urged towards each other when a force is applied to the first saddle and the second saddle in a distal direction. At least one of the branches can have a barb extending out from the at least one branch, and the barb can be adapted to engage with a mating lip of the proximal base to prevent the clamp from moving in a proximal direction relative to the proximal base after the barb is engaged with the mating lip.

In an embodiment, a fixation system for knotlessly stabilizing anatomical structures can include a flexible member, and a proximal button assembly with one or more saddles, and the one or more saddles can be adapted for the flexible member to be wrapped around the one or more saddles to form two or more loops around the one or more saddles and an anatomical structure of a patent. The system can include a first protuberance having a first jaw, and a second protuberance having a second jaw, and the first jaw and the second jaw can defining a clamping area, wherein the flexible member can be adapted to be passed through around an anatomical structure of a patient to form a loop through an anatomical structure, and wherein the flexible member can be adapted to be passed through the clamping area of the proximal button assembly, and the clamping area can be adapted to clamp onto the flexible member and lock the flexible member in the clamping area. The one or more saddles can be operatively connected to the first protuberance and the second protuberance, whereby a force applied in a distal direction to the one or more saddles by the flexible member causes the first protuberance and the second protuberance to be urged together, thereby closing the clamping area around the flexible member.

The fixation system can include a proximal base having a pocket configured to accommodate the protuberances, the pocket having an opening to allow the one or more saddles and the flexible member to extend through the opening in a distal direction. The proximal base can include a sloped mating ramp on an interior of the pocket, the sloped mating ramp configured to engage with the protuberances and urge the protuberances towards each other when a force is applied to the one or more saddles in a distal direction. The system can include a distal button with a pair of openings or with at least one opening or eyelet adapted for the flexible member to be routed through the distal button as part of the two or more adjustable loops spanning the distance between the distal and proximal buttons and that may pass through, or between or around anatomical structures.

In an embodiment, a method of stabilizing anatomical structures without the need for knots can include providing a flexible member and providing a proximal button with one or more saddles, and the one or more saddles can be adapted for the flexible member to be wrapped around the one or more saddles. The proximal button can include a first protuberance having a first jaw, and a second protuberance having a second jaw, and the first jaw and the second jaw can define a clamping area adapted to clamp onto a flexible member and lock the flexible member in the clamping area without the need for tying knots. The one or more saddles can be operatively connected to the first protuberance and the second protuberance, whereby a force applied in a distal direction to the one or more saddles by the flexible member can cause the first protuberance and the second protuberance to be urged together, thereby closing the clamping area around the flexible member. The method can include passing the distal button and flexible member through a bore or an opening or a gap in an anatomical structure of a patient, flipping the distal button to brace against anatomical structure and to prevent the distal button from being able to be pulled back through said bore, pulling on the free ends of the flexible member through the clamping area between the first jaw and the second jaw in a proximal direction to tension the loops, pulling on the free ends of the flexible member so that the tightening (shortening or tensioning) loops apply compressive force to the structures between the distal and the proximal buttons, thereby urging the first and second protuberances onto the proximal base and clamping the jaws together to lock the flexible member in place, and releasing the tension on the free ends of the flexible member, resulting in the proximal button assembly locking the flexible member in place without the need for tying knots.

### BRIEF DESCRIPTION OF THE DRAWINGS

The figures which accompany the written portion of this specification illustrate embodiments and method(s) of use for the present invention, Devices and Systems for the Stabilization of Bones, Tissues, and Grafts, constructed and operative according to the teachings of the present invention:

FIG. 1 is a perspective view of a stabilization system that can be used for bones, tissues, and grafts, according to an illustrative embodiment;

FIG. 2 is another perspective view of the stabilization system from a different perspective, showing a proximal button assembly, flexible member, and a distal button, according to an illustrative embodiment;

FIG. 3A is an exploded perspective view of a proximal button assembly, showing a clamp and proximal base, according to an illustrative embodiment;

FIG. 3B is a perspective view of a proximal button assembly with locking barbs on the clamp, according to an illustrative embodiment;

FIG. 4 is a perspective view of a distal button with an optional recess in the distal surface, according to an illustrative embodiment;

FIG. 5 is a perspective view of an extended footprint washer for a proximal base, according to an illustrative embodiment;

FIG. 6 is a perspective view of a stabilization system with an extended footprint washer on the proximal base, according to an illustrative embodiment;

FIG. 7 is a perspective view of a stabilization system with an extended footprint washer and without a distal button, according to an illustrative embodiment;

FIG. 8 is a perspective view of a stabilization system using two proximal button assemblies, according to an illustrative embodiment;

FIG. 9 is a perspective view of a stabilization system with one end of the flexible member tied to the distal button, according to an illustrative embodiment;

FIG. 10 is a perspective view of a stabilization system with the flexible member doubled over through the stabilization system, according to an illustrative embodiment;

FIG. 11A is a perspective view of a proximal base with a continuous annular ramp, according to an illustrative embodiment;

FIG. 11B is a perspective view of an axially symmetric proximal base with a continuous annular ramp, according to an illustrative embodiment;

FIG. 12 is a perspective view of a clamp having an eyelet above the saddle for routing the flexible member through the clamp, according to an illustrative embodiment;

FIG. 13 is a perspective view of a clamp having an eyelet and a narrow distal tip below the saddle, according to an illustrative embodiment;

FIG. 14 is a perspective view of a stabilization system using a two-piece clamp with side-by-side branches, according to an illustrative embodiment;

FIG. 15 is a partially-cut away view of the distal button assembly using the two-piece clamp with side-by-side branches of FIG. 14, showing inner workings, according to an illustrative embodiment;

FIG. 16 is a perspective view of a proximal button assembly using a two-piece clamp with stacked branches, according to an illustrative embodiment;

FIG. 17 is a perspective view of a stabilization system incorporating the proximal button assembly of FIG. 16 using the two-piece clamp with stacked branches, according to an illustrative embodiment;

FIG. 18 is a perspective view of a proximal button assembly that operates without a proximal base and using a two-piece clamp with stacked branches, according to an illustrative embodiment;

FIG. 19 is a perspective view of a stabilization system incorporating the proximal button assembly of FIG. 18 that operates without a proximal base and using a two-piece clamp with stacked branches, according to an illustrative embodiment;

FIG. 20 is a side view of the stabilization system of FIG. 19 without a proximal base, shown with distal mating surfaces of the clamp engaged with another mating surface, according to an illustrative embodiment;

FIG. 21 is a perspective view of a one-piece clamp without a proximal base, according to an illustrative embodiment;

FIG. 22 is a side view of the one-piece clamp of FIG. 21 without a proximal base, according to the illustrative embodiment;

FIG. 23 is a side view of a stabilization system utilizing the one-piece clamp without a proximal base of FIG. 21, according to an illustrative embodiment;

FIG. 24 is a diagram of a method of assembling a stabilization system that can be used to stabilize an anatomical structure during a surgery without tying a knot (free from knots), according to an illustrative embodiment; and

FIG. 25 is a diagram of a method of using a stabilization device to stabilize an anatomical structure during surgery, according to an illustrative embodiment.

### DETAILED DESCRIPTION

There are a great many possible implementations of the present stabilization system, too many to describe herein. Some possible implementations are described below. In the following description, for purposes of explanation, numerous specific details are set forth in order to provide a thorough understanding thereof. It should be clear, however, that the innovation can be practiced without various specific details. Various embodiments are discussed hereinafter. It should be noted that the figures are described only to facilitate the description of the embodiments. They are not intended as an exhaustive description of the invention and do not limit the scope of the invention. Additionally, any particular embodiment need not have all the aspects or advantages described herein. Thus, in various embodiments, any of the features described herein from different embodiments may be combined. It cannot be emphasized too strongly, however, that these are descriptions of implementations of the invention, and not descriptions of the invention, which is not limited to the detailed implementations described in this section but is described in broader terms in the claims.

The devices and systems described herein for the stabilization of bones, tissues, and grafts can include a proximal button assembly, a distal button, and a flexible member. The flexible members can include a suture, tape, cable, cord, wire, rope, monofilament, chain, or other flexible members. The proximal button assembly can include a proximal base and a clamp. In various embodiments, the stabilization system can include a proximal button assembly and a distal button connected by a flexible member. The system can be assembled with the flexible member wrapped around or passed through the proximal button assembly and distal button to form at least two but more preferably three or more adjustable loops of the flexible member between the proximal button assembly and the distal button. The distance between the proximal button assembly and the distal button can be decreased by shortening the loops, which can be accomplished by pulling on the free ends of the flexible member. When a pull force is applied to the free end(s) of the flexible member, the distal button is urged towards the proximal button assembly, which in turn applies compression force to the boney anatomy therebetween. When the pull force to the free ends of the flexible member is released, the clamp can flex inward and pinch the flexible member near the free ends to lock the device in its current state and preventing the distal button from moving away from the proximal button assembly.

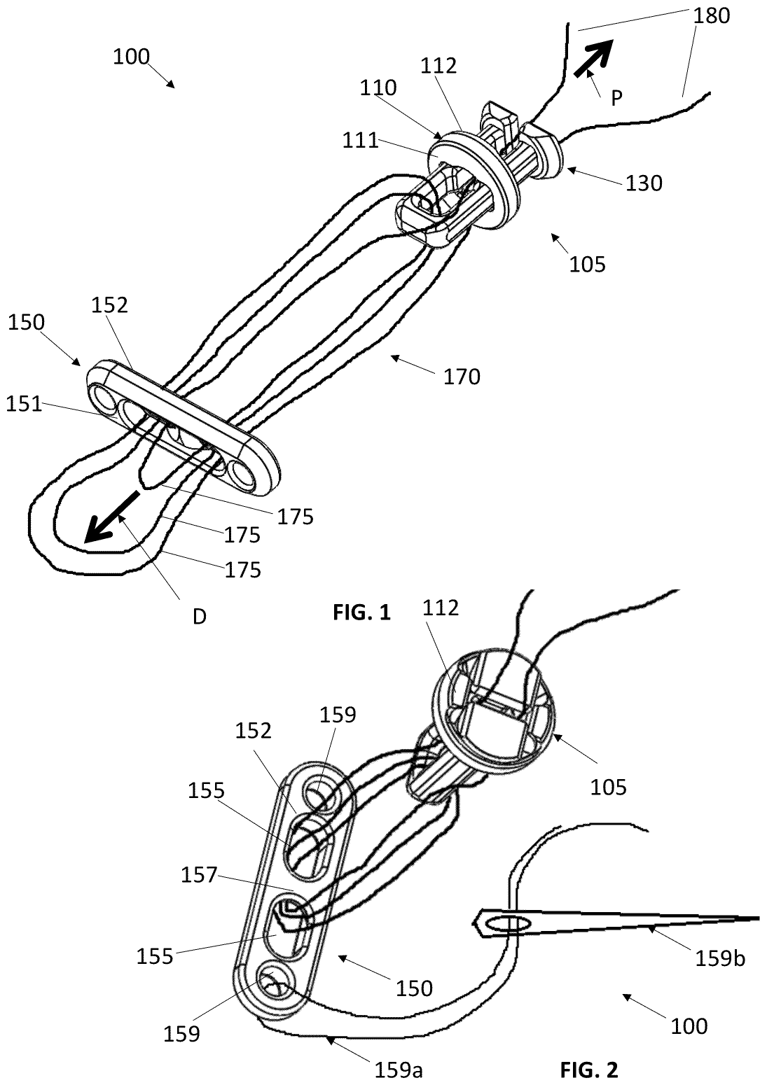

FIG. 1 is a perspective view of a stabilization system that can be used for bones, tissues, and grafts, according to an illustrative embodiment, and FIG. 2 is another perspective view of the stabilization system from a different perspective, according to an illustrative embodiment. The stabilization system 100 can include a distal button 150, and a proximal button assembly 105. Distal button 150 can include a distal surface 151, a proximal surface 152, a pair of medial openings 155 separated by isthmus 157, and a pair of optional side openings 159. Openings 159 can be optional and in various embodiments there may be zero, one, two, three, four or more side openings 159. Side openings may also serve as attachment points for optional passing sutures 159a, which may be either pre-assembled or added intraoperatively; the optional passing sutures 159a (shown in FIG. 2) may be further attached to an optional passing needle 159b (shown in FIG. 2), which may be pre-assembled to the passing suture or added intraoperatively. The passing needle and suture can be used to pass through a tunnel in the bone and puncture the skin distal of the surgical access side, and can be used to pull the distal button through the bone tunnel so that it can be flipped 90 degrees to brace against bone on the opposite side of the bone tunnel thereby allowing it to resist tension applied to a flexible member 170. The proximal button assembly 105 can include a clamp 130 and a proximal base 110. The proximal base 110 can have a distal surface 111 and a proximal surface 112. The flexible member 170 can form loop(s) between the distal button 150 and proximal button assembly 105 to form the stabilization system 100.

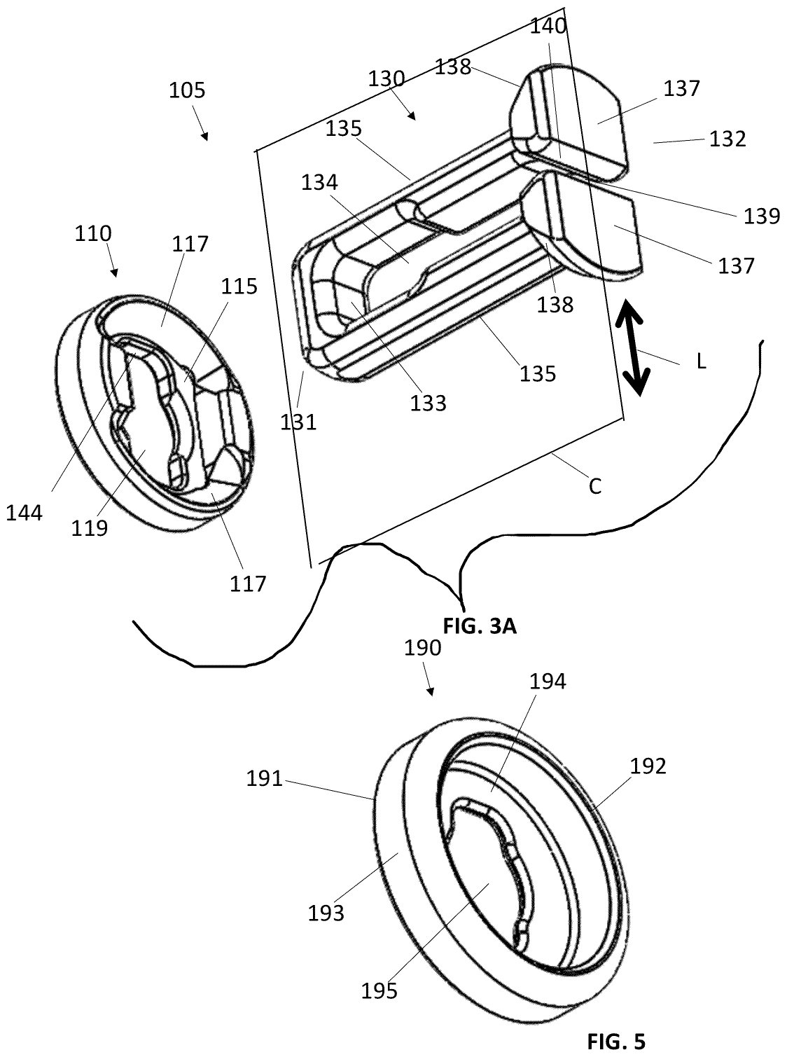

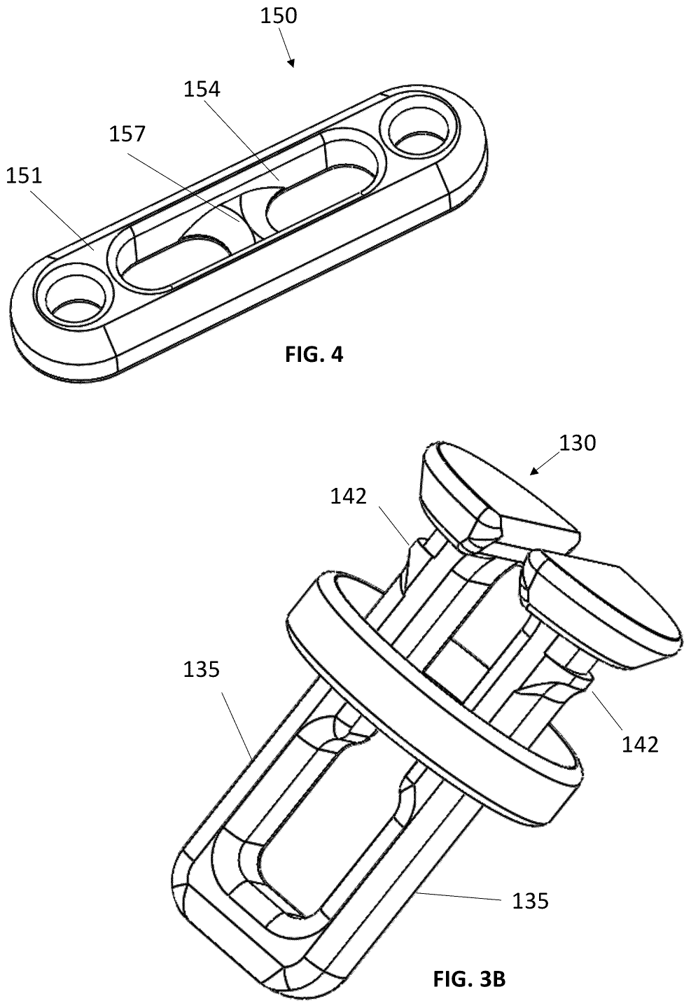

FIG. 3A is an exploded perspective view of a proximal button assembly, showing a clamp and proximal base, according to an illustrative embodiment, and FIG. 3B is a perspective view of a proximal base, according to an illustrative embodiment. The proximal base can have a pocket 115 having sloped (or curved, ramped, canted, sloping, etc.) ramps 117 and a through opening 119. The clamp 130 can have a distal end 131, a proximal end 132, a saddle 133 located at the distal end of the clamp, and two branches 135 extending from the saddle. The branches 135 can have protuberances 137 at the proximal ends of the branches. Each protuberance 137 can have a clamping jaw 140. The space between the protuberances 137 can form a clamping area 139, and the two clamping jaws 140 of the two protuberances 137 can be brought together to clamp onto the flexible member within the clamping area 139. A coronal plane C, also referred to as a frontal plane, as shown on FIG. 3A, can be thought of as extending along the clamp lengthwise in a proximal to distal direction, and laterally side to side. As shown in FIG. 3A, the branches can move towards each other in a lateral direction along the coronal plane, along lateral direction arrow L.

The protuberances 137 can have force receiving areas 138 that can be adapted to receive forces that can press the protuberances together. In various embodiments, force receiving areas 138 can be sloped (or curved, ramped, canted, sloping, etc.) mating surfaces configured to mate with sloped mating ramps 117 of the proximal base 110. Pressing the force receiving areas 138 against sloped mating ramps 117 can cause the branches 135 to flex toward each other, resulting in pinching of a flexible member in the clamping space 139.

Turning now to FIGS. 1, 2, and 3A, the flexible member 170 can be assembled with the distal button and the proximal button assembly by passing it at least once (and more preferably twice) through an opening 134 above the saddle 133 of the clamp 130 and at least twice (and more preferably three times) through each of the medial openings 155 of the distal button, and then feeding the two free ends of the flexible member in the proximal direction through the opening 119 of the proximal base 110 and through the clamping space 139 of the clamp 130. This result can result in at least 1 (and more preferably 2 and most preferably 3 or more) adjustable loops between the proximal button assembly 105 and the distal button 150. The adjustable loops can be shortened by pulling on the free ends 180 of the flexible member 170 in the proximal direction, shown by arrow P. Pulling on the free ends 180 of the flexible member 170 in the proximal direction, also referred to as the tightening direction, can result in the adjustable loops being shortened and/or the distal button being urged toward the proximal button assembly, which can result in the application of compression force to any bony anatomy and/or other tissues between the distal button and the proximal base.

Put another way, at this stage the distal button 150 and the proximal base 110 are applying forces to the tissues that are between the distal button and the proximal button assembly. At the same time, elastic resistance forces or restoring forces generated in the tissues that are being reduced (or compressed) along with external forces and other biomechanically relevant forces, may act in the direction that would, if unconstrained and unresisted,—urge the buttons away from each other.

Once the pull force on the free ends is released, the residual tension in the adjustable loops of the flexible member and/or the residual compressive stress in the anatomical structures between the distal and proximal buttons, can cause the sloped surfaces 138 of the clamp to press against the sloped ramps 117 of the proximal base, in turn causing the clamp branches 135 to deflect toward each other, reducing the width of clamping space 139 and pinching the flexible member where its ends are passing through the clamping space. This clamping of the flexible member 170 in the clamping space 139 between the jaws 140 can cause a locking action which can prevent the flexible member from being pulled backwards and allowing the loops to elongate. That is to say, clamping on the ends of the flexible member can prevent the adjustable loops of the flexible member from lengthening, and preventing the adjustable loops from lengthening can resist the distal button from moving away from the proximal button assembly.

The adjustable loops between the clamp and the distal button can act analogously to a compound pulley system, which is to say that with each additional loop, the mechanical advantage (e.g. difference between the pull force on the free end of the flexible member and the force generated by the resulting proximally-directed movement of the distal button) may increase, but so does the friction generated between the flexible member and the rigid components. It should be noted that in various embodiments, the locking behavior of the stabilization system may rely on the proximal base being braced against a proximal counterforce surface (such as bone or other tissue, or the surface of a metal or polymeric or composite fracture plate or biomedical device, or other mating surface or bracing surface or provider of Newtonian counterforce), with the distal button being pulled up against and being braced against a distal counterforce surface (such as bone or other tissue, fixation plate, interference screw or other biomedical device, or other mating surface, or bracing surface, or provider of Newtonian counterforce). In such a loading environment, pulling on the free end of the flexible member causes the adjustable loops to pull on the clamp 130, which in turn applies distally-directed load to the proximal base 110 in the distal direction shown by arrow D, which in turn applies load to the proximal counterforce surface (e.g. bone or biomedical device). When the clamp pulls against the proximal base in the distal direction D, the force receiving areas of the clamp may forcefully engage the sloped ramp of the proximal base, causing the clamp branches to deflect toward each other and clamp or bite down on the ends of the flexible member, which prevents lengthening of the loops and acts to inhibit the distal button from moving away from the proximal base. Furthermore, the stabilization system ideally operates with a mechanical advantage such that a pull force applied to the free end of the flexible member results in a greater force urging the distal button toward the proximal base. In this case, a counterforce applied by the tissues between the buttons that is applied directly to the distal button and directed to urge it away from the proximal base can be resisted by a (preferably significantly) smaller force applied to the free end of the flexible member.

In such a loading scenario, pulling on the free ends of the flexible member both urges the distal button toward the proximal base and causes the clamp to bite down on the free ends of the flexible member. This clamping of the flexible member can work to resist additional pulling of the free ends through the clamping area, and thereby resisting further proximal movement of the distal button desired by the user. However, because of the mechanical advantage, the pull force applied to the free ends of the flexible member can overcome at least some amount of this resistance (or locking force) caused by the clamping action, and can continue to urge the distal button toward the proximal base and to thereby cause the stabilization system to apply additional compressive load to the anatomical structures disposed between the distal button and the proximal base and also to apply additional tension to the loops of the flexible member.

Once the pull force to the free ends of the flexible member is released, the residual compressive stress in the compressed tissues as well as any other anatomically or biomechanically relevant forces could attempt to lengthen the flexible member loops and force the distal button away from the proximal base, but that force would be acting at a mechanical disadvantage as described above, so the locking force generated by the clamp biting down on the free ends of the flexible member may (ideally) be sufficient to resist further movement of the distal button away from the proximal button assembly. The tension achieved in the flexible member can be maintained, which can maintain a compressive force on the structures disposed between the distal button and the proximal base. Thereby keeping them in the desired alignment or orientation or spacing. The stabilization system is then considered “locked” and without further need of tying knots to maintain tension in the flexible member or relative position of distal and proximal buttons.

FIG. 3B is a perspective view of a proximal button assembly with locking barbs on the clamp, according to an illustrative embodiment. The clamp 130 can optionally include a barb or a pair or barbs 142 on the clamp branches 135. When the branches of the clamp get deflected toward each other during the clamping action, the barbs can pass underneath a mating lip 144 or lips in the proximal base. The barbs 142 can then click in and be detained under the mating lip of the proximal base once the clamping action and branch deflection is lessened or released. This locking barb 142 can secure the clamp and proximal base together in the fully engaged position, and can prevent the clamp from moving in a proximal direction relative to the proximal base.

FIG. 4 is a perspective view of a distal button with an optional recess in the distal surface, according to an illustrative embodiment. The distal button 150 can optionally include a recess 154 located in the distal surface 151. The optional recess 154 can accommodate the thickness of the flexible member when the flexible member is wrapped around the isthmus 157, and can reduce the overall prominence or thickness of the device on the distal side.

FIG. 5 is a perspective view of an extended footprint washer for a proximal base, according to an illustrative embodiment. The stabilization system 100 can optionally include an extended footprint washer 190. The washer 190 can include a distal surface 191, a proximal surface 192, an outer surface 193, a washer recess 194 in the proximal surface and an opening 195 in communication between proximal to distal surfaces. The washer recess 194 is sized in such a way so as to allow the proximal button assembly to be disposed within the washer recess 194 and the opening 195 may be sized such that distal button 150 could pass through it for assembly, disassembly and general versatility purposes. The washer 190 can increase the area of contact between the proximal base 110 of the proximal button assembly 105 and the anatomical structure or other proximal counterforce surface that the proximal base presses against. This can thereby reduce stresses exerted on the anatomical structure (or fracture plate, suture anchor, interference screw or any other structure) by distributing the forces applied by the proximal base to the anatomical structure over a greater contact area.

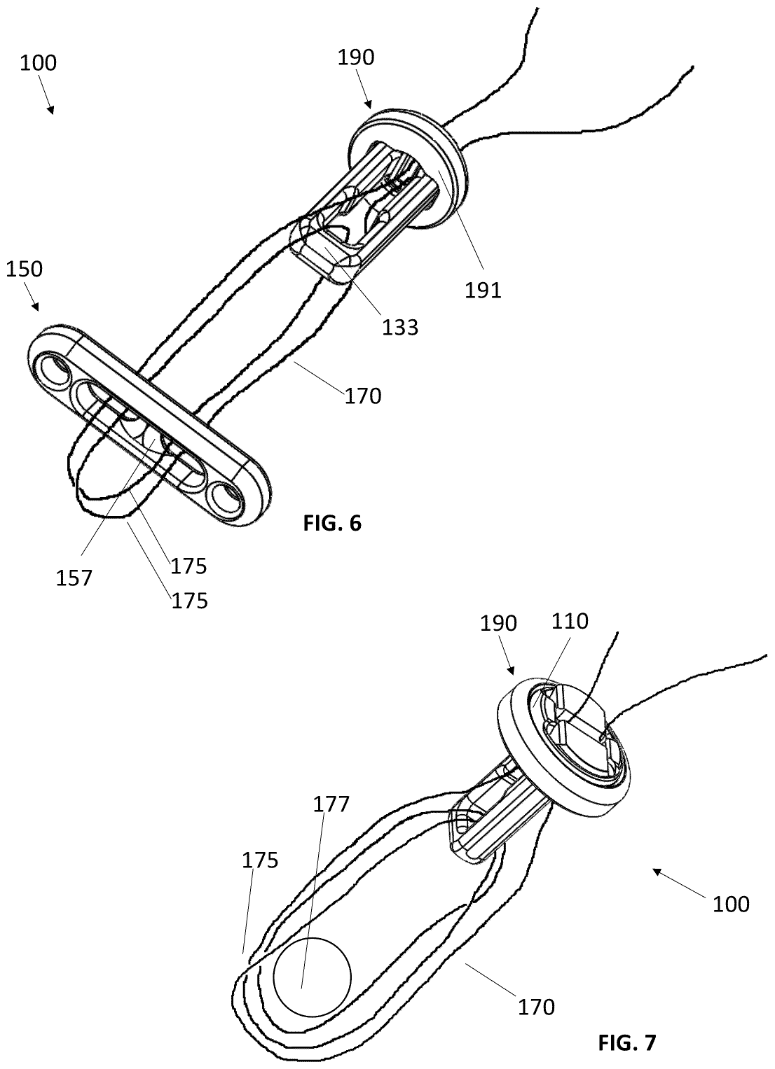

FIG. 6 is a perspective view of a stabilization system with an extended footprint washer on the proximal base, according to an illustrative embodiment. The proximal base can be held within the recess 194 of the extended footprint washer. Because the distal surface 191 of the extended footprint washer is larger than the distal surface 111 of the proximal base, adding the optional extended footprint washer 190 to the proximal base can have the effect of increasing the contact surface are of the proximal base against the anatomical structures, thereby reducing the stress exerted on the anatomical structure.

The embodiment shown in FIG. 6 is depicted with only two loops 175 in the flexible member 170. The assembly can have two loops, three loops or more. Having two loops (compared to the three loops as shown in FIG. 1) can decrease the tensile strength of the assembly due to the decreased number of strands of flexible member crossing the span between the proximal and distal sides of the assembly. However, having fewer loops can also decrease the friction due to having fewer turns of the flexible member that need to slide around the clamp saddle 133 and isthmus 157 when the free ends are pulled.

FIG. 7 is a perspective view of a stabilization system without a distal button, according to an illustrative embodiment. Proximal base 110 is shown within the recess of the expanded footprint washer 190, so that the washer 190 can increase the footprint of the proximal base 110. The clamp 130 is shown in a clamped conformation, with the flexible members held securely within the clamping area 139 between the jaws 140.

In various embodiments, the distal button can be optional. The loops 175 of the flexible member 170 can be wrapped around and/or through an anatomical securement point such as a tendon, tendon graft, bone, bone graft, or other distal securement point 177. In other embodiments, an anatomical structure or soft tissue or soft tissue graft or bone graft can simply be passed through or inserted into the loops of the flexible member in any embodiment, either with or without the distal button being present as part of the stabilization device. In various embodiments, two stabilization systems can be assembled together, with the loops 175 of the first stabilization system wrapped through the loops 175 of the second stabilization system, so that the loops of one stabilization system can act as the distal securement point 177 of the other stabilization system and vice versa.

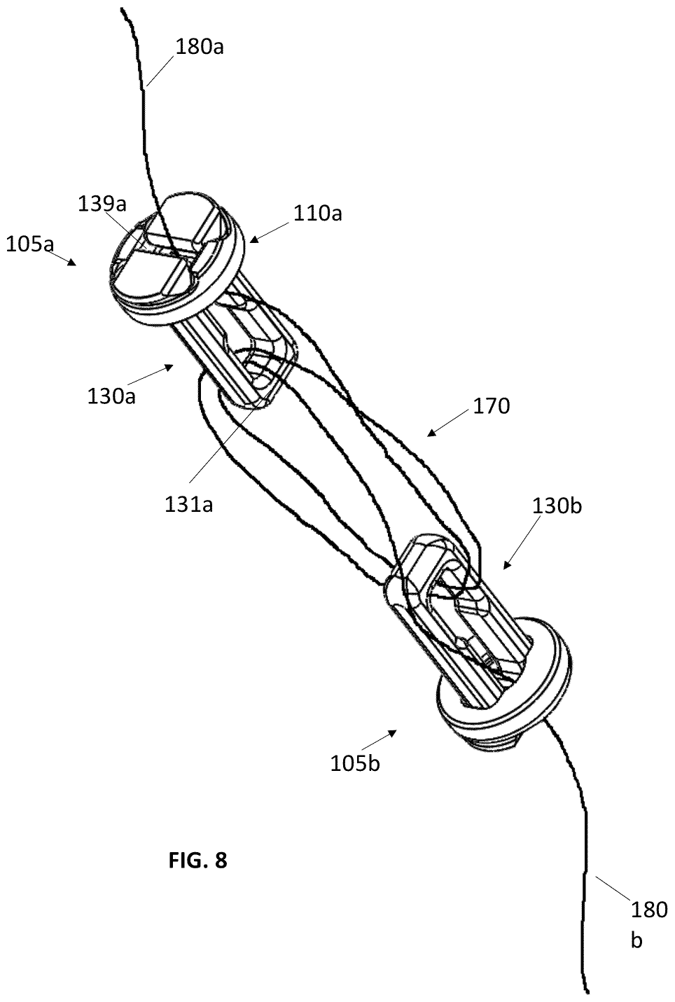

FIG. 8 is a perspective view of a stabilization system using two proximal button assemblies, according to an illustrative embodiment. In various embodiments, two proximal button assemblies, 105a and 105b can be arranged end to end, and the flexible member can be wrapped around the saddle of each proximal button assembly. The saddle 133a of one proximal button assembly 105a can act as the distal securement point of the other proximal button assembly 105b, and vice versa. The flexible member can be wrapped around each saddle to form two or more loops between the two saddles. In various embodiments, a first free end 180a can extend through the clamping area 139a of a first proximal button assembly 105a, and a second free end 180b can extend through the clamping area of a second proximal button assembly 105b. A user can then pull the free ends 180a and 180b away from each other, and in doing so, can draw the proximal button assemblies 105a and 105b closer together, thereby creating a compressive force on the anatomical structures between proximal buttons 105a and 105b to stabilize the anatomical structures between the proximal buttons. As the pulling force on the free ends 109a, 108b is released, each end 180a, 108b of the flexible member 170 is clamped within the clamping area of each proximal button assembly, thereby locking the proximal button assemblies 105a, 105b in place and preventing them from moving away from each other.

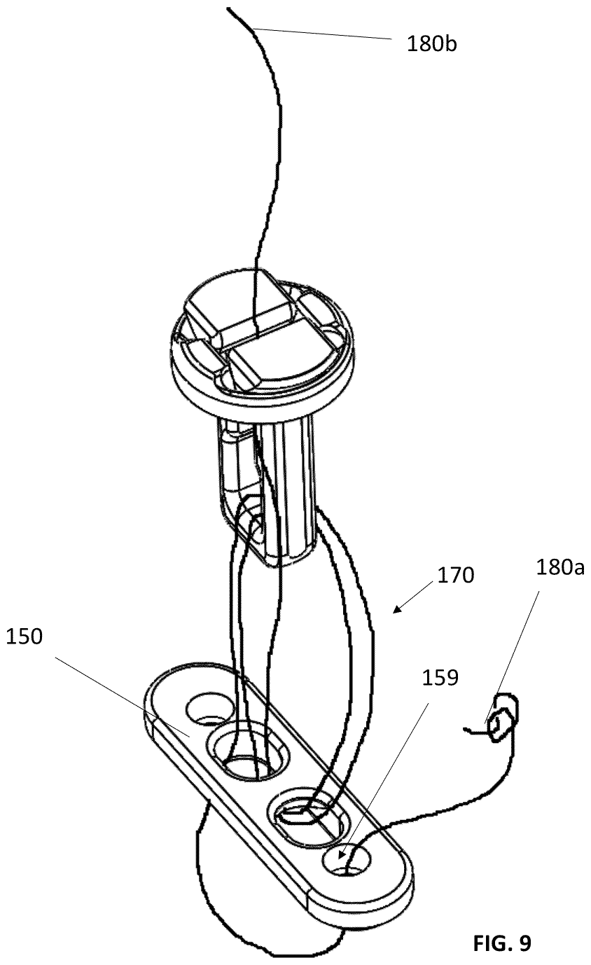

FIG. 9 is a perspective view of a stabilization system with one end of the flexible member tied to the distal button, according to an illustrative embodiment. In various embodiments, the first end 180a of the flexible member can be tied to the distal button 150. In various embodiments, the first end 180a can be tied through a side opening 159. A user can pull on free end 180b to tighten the stabilization system around the anatomical structures while free end 180a is anchored to the distal button 150.

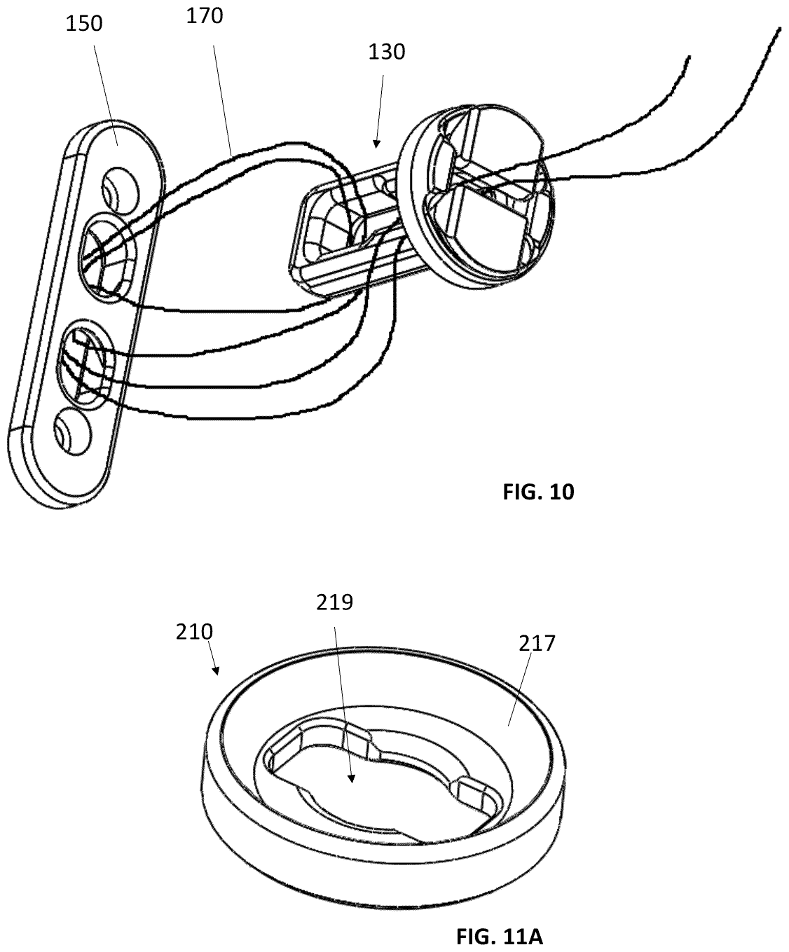

FIG. 10 is a perspective view of a stabilization system with the flexible member doubled over through the stabilization system, according to an illustrative embodiment. In various embodiments, one end of the flexible member can pass through both openings 155, and the flexible member is thereby folded around (or wrapped around or bent around) the isthmus 157 of the distal button with both free ends pointed proximally. The free ends of the flexible member 170 can then be held together and, together, can be fed at least once through the clamp opening 134 and at least once through each of the openings 155 and at least once through the clamping space 139. Flexible member 170 can be looped through the system to create different loops that travel different paths through the distal button 150 and the clamp 130. In various embodiments, the flexible member can be looped around or passed through the parts of the system in various different routes and can create various loops that may or may not be the same.

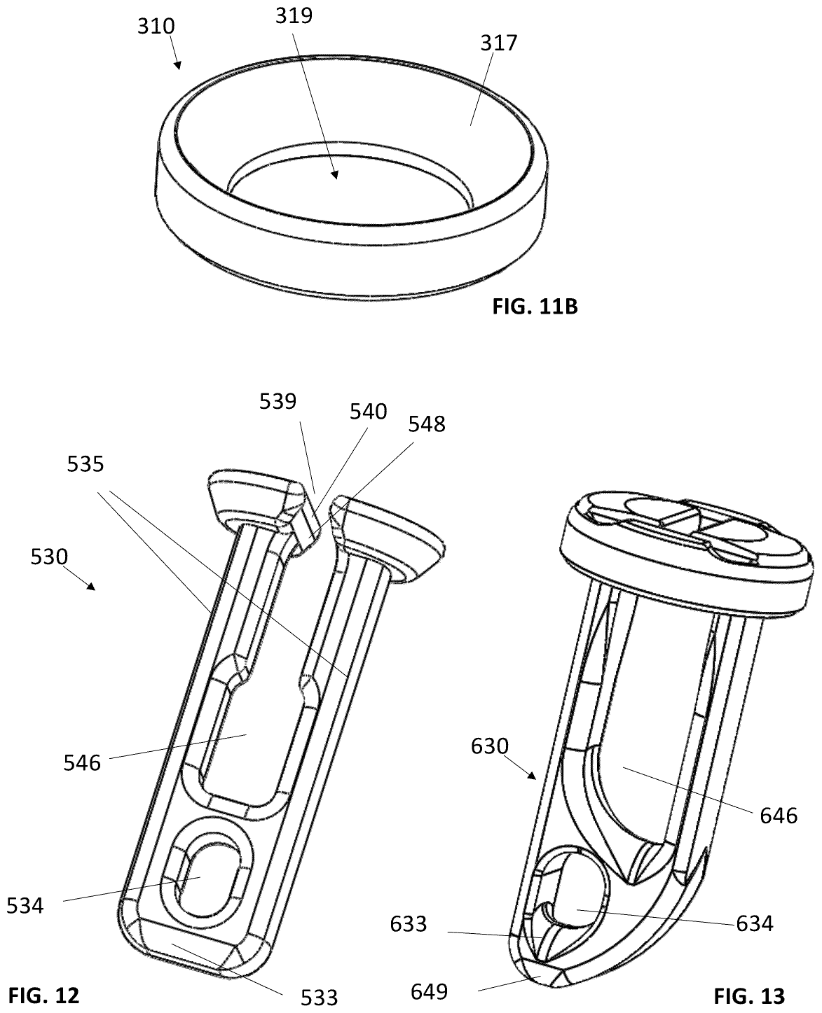

FIG. 11A is a perspective view of a proximal base with a continuous annular ramp, according to an illustrative embodiment. Proximal base 210 can have a continuous annular ramp 217 that can have the shape of the inner surface of a truncated cone. Proximal base 210 can be used interchangeably with a proximal base 110 in a proximal button assembly 105. The continuous annular ramp 217 can engage both the of the force receiving areas 138 of the two branches 135. Similar to the proximal base 110 shown in FIG. 3A, forcing the force receiving areas 138 against the continuous annular ramp 217 can cause the branches 135 to flex toward each other, resulting in pinching of any flexible member in the clamping space 139.

The opening 219 can be the same as the opening 119 of FIG. 3A, and can allow the clamp and the flexible member to pass through the proximal base 210. The non-axially symmetric shape of the opening 219 prevents the clamp from rotating within the proximal base 210.

FIG. 11B is a perspective view of an axially symmetric proximal base with a continuous annular ramp, according to an illustrative embodiment. Proximal base 310 can have a continuous annular ramp 317 that can have the shape of the inner surface of a truncated cone. Proximal base 310 can be used interchangeably with a proximal base 110 in a proximal button assembly 105. The continuous annular ramp 317 can engage both the of the force receiving areas 138 of the two branches 135. Similar to the proximal base 110 shown in FIG. 3A, forcing the force receiving areas 138 against the continuous annular ramp 317 can cause the branches 135 to flex toward each other, resulting in pinching of any flexible member in the clamping space 139.

The opening 319 can allow the clamp and the flexible member to pass through the proximal base 310. The axially symmetric shape of the opening 319 can allow the clamp to rotating within the proximal base 310. The axially symmetric shape of the opening 319 can also allow a surgeon to insert the clamp into the proximal base during surgery without needing to account for the axially rotational position of the clamp relative to the proximal base 310, thereby potentially making surgeries easier and faster. Other potential benefits of using a continuous annular ramp arrangement can include increased strength, improved machineability and reduced cost of the component.

FIG. 12 is a perspective view of a clamp 530 having a dedicated separate opening, also referred to as an eyelet, 534 above the saddle 533 for routing the flexible member through the clamp 530, according to an illustrative embodiment. Clamp 530 can have a first opening that can be an eyelet 534, and can have a separate open space 546 between the branches 535. The open space 546 allows the branches 535 to flex towards each other when the clamp is pulled into the proximal base 110/120/130, thereby allowing the jaws 540 to be urged together to close the clamping area 539 around the flexible member. In various embodiments, the jaws 540 can have a tapered jaw surface 548. Tapered jaw surface 548 can allow the flexible member to more easily slide through the clamping space in the proximal (tightening) direction than in the distal (loosening) direction, so that the loops of the flexible member can be tightened more easily than they can be loosened.

The dedicated separate opening, or eyelet 534 for the flexible member can allow the flexible member to be routed securely through the clamp 530 in a way that the flexible member cannot fall out of the clamp. During the surgical procedure, the clamp 530 can hang from the flexible member through the eyelet 534 without falling away from the flexible member. Routing one or more of the flexible member loops through a separate opening in the clamp may also help reduce friction and make tightening (reduction) process easier.

FIG. 13 is a perspective view of a clamp having an eyelet and a narrow distal tip below the saddle, according to an illustrative embodiment. In various embodiments, the clamp 630 can taper to a narrow distal tip 649 at the distal end. This can allow the clamp to be more easily handled and inserted into the proximal base. The clamp 630 can include an eyelet 634 above the saddle 633 so that the flexible member can be securely threaded through the eyelet 634. In various embodiments, the distal tip 649, the eyelet 634, and the open space 646 can be irregularly shaped or laterally asymmetrical along the coronal plane on either side of an axial-distal central axis. This arrangement may help reduce friction between adjacent flexible member loops (or passes or sections or portions).

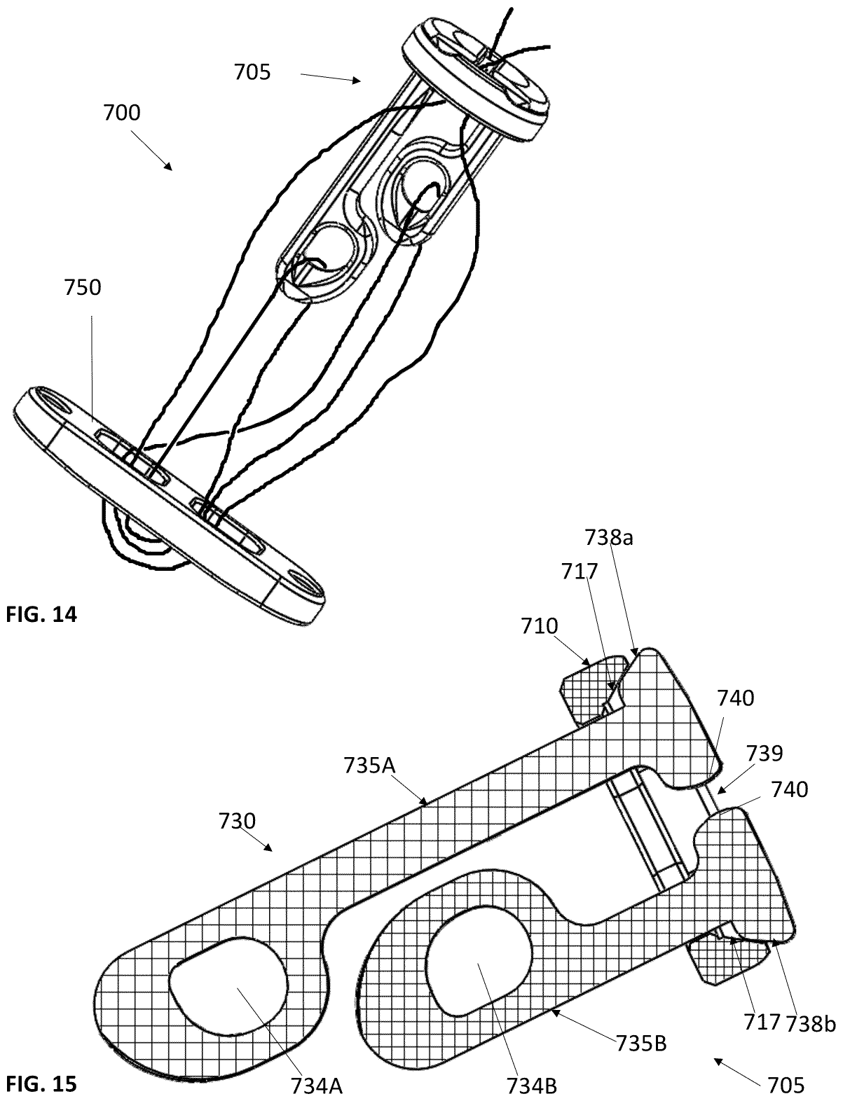

FIG. 14 is a perspective view of a stabilization system using a two-piece clamp with side-by-side branches, according to an illustrative embodiment, and FIG. 15 is a partially-cut away view of the distal button assembly using the two-piece clamp with side-by-side branches of FIG. 14, showing inner workings, according to an illustrative embodiment. In various embodiments, a stabilization system 700 can use a two-piece clamp 730, and the two-piece clamp can include two branches 735a and 735b that can be separate components, positioned next to each other in a side-by-side arrangement. Each of the branches 735a and 735b can have an eyelet 734a and 734b that can accommodate the flexible member, and the two eyelets can be laterally and/or axially distant from each other. The flexible member can be passed through the distal button and at least once through each of the eyelets 734a, 734b of the branches 735a and 735b, to create at least two loops between the proximal button assembly 705 and the distal button 750.

The proximal clamp 730 does not need to flex (or elastically or plastically deform) to allow the jaws 740 to come together and close the clamping area 739. As the flexible member is pulled tight and the force receiving areas 738a, 738b that can be sloped mating surfaces are pulled against the sloped mating ramps 717 of the proximal base 710, the two separate side-by-side branches 735a and 735b can move closer together. As the flexible member is pulled tight and the force receiving areas 738a, 738b are pulled against the sloped mating ramps 717 of the proximal base 710, the jaws 740 can be urged towards each other to bite down on the flexible member and lock the flexible member in place. Proximal clamp 730 can be free of flexing or deformation while still allowing the jaws to bite down on the flexible member and lock the flexible member in place when the flexible member is pulled tight in the proximal direction.

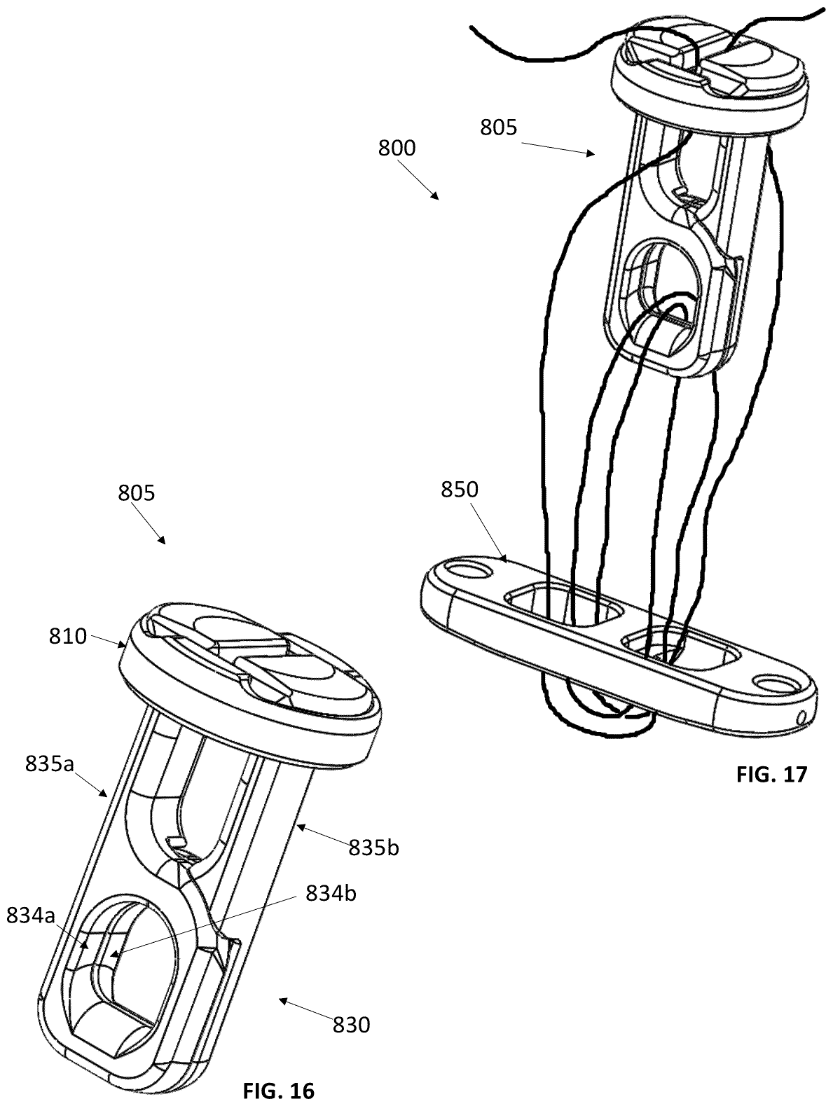

FIG. 16 is a perspective view of a proximal button assembly using a two-piece clamp with stacked branches, according to an illustrative embodiment, and FIG. 17 is a perspective view of a stabilization system incorporating the proximal button assembly of FIG. 16 using the two-piece clamp with stacked branches, according to an illustrative embodiment. In various embodiments, a stabilization system 800 can use a two-piece clamp 830, and the two-piece clamp can include two branches 835a and 835b that can be separate components, positioned next to each other in a stacked arrangement. Each of the branches 835a and 835b can have an eyelet 834a and 834b that can accommodate the flexible member. In a stacked conformation, the eyelets 834a and 834b can be stacked together, so that the flexible member can pass through both openings 834a, 834b together in each loop. As shown in FIG. 17, the flexible member passes through the distal button 850 and both 834a and 834b in each loop.

The proximal clamp 830 does not need to flex (or elastically or plastically deform) to allow the jaws to come together and close the clamping area. As the flexible member is pulled tight and the force receiving areas are pulled against the sloped mating ramps of the proximal base 810, the jaws can be urged towards each other to bite down on the flexible member and lock the flexible member in place. As the flexible member is pulled tight and the force receiving areas are pulled against the sloped mating ramps of the proximal base 810, the two separate stacked branches 835a and 835b can slightly rotate relative to each other about the stacked openings 834a and 834b as the jaws come together. Proximal clamp 830 can be free of flexing or deformation while still allowing the jaws to bite down on the flexible member and lock the flexible member in place when the flexible member is pulled tight in the proximal direction.

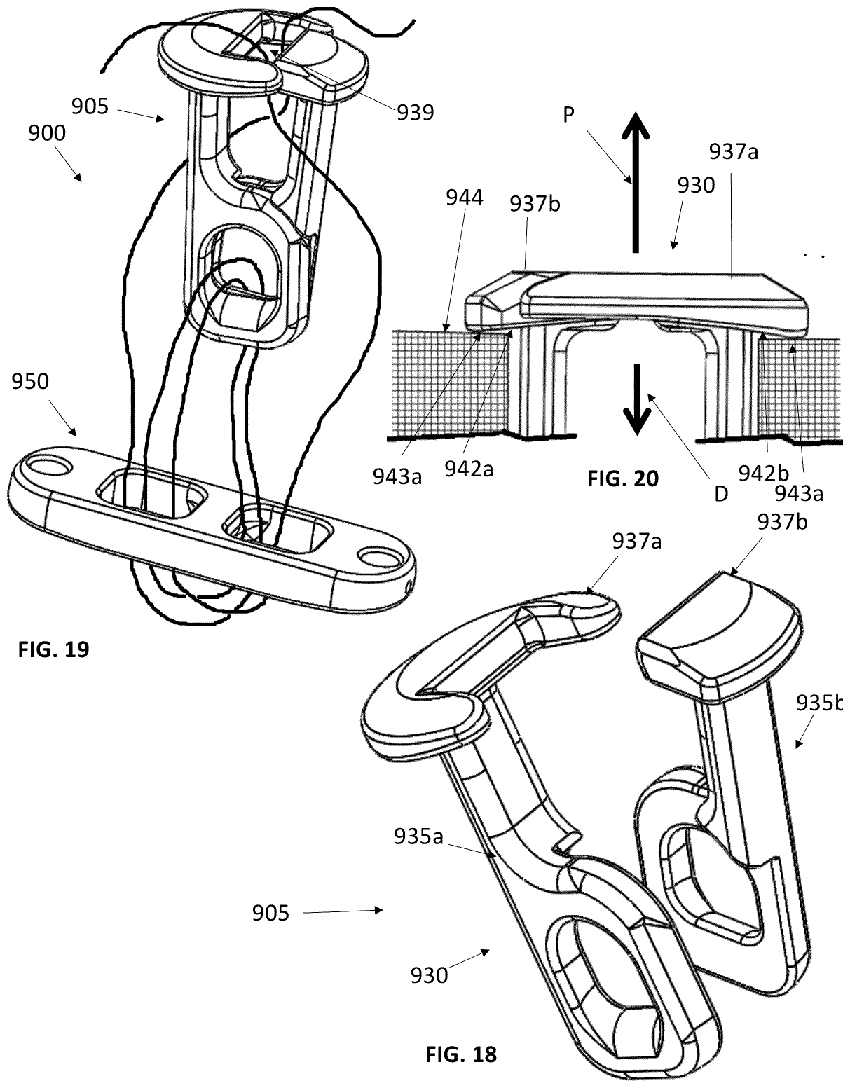

FIG. 18 is a perspective view of a proximal button assembly that operates without a proximal base and using a two-piece clamp with stacked branches, according to an illustrative embodiment, and FIG. 19 is a perspective view of a stabilization system incorporating the proximal button assembly of FIG. 18 that operates without a proximal base and using a two-piece clamp with stacked branches, according to an illustrative embodiment. In various embodiments, a stabilization system 900 can use a two-piece clamp 930, and the two-piece clamp can include two branches 935a and 935b that can be separate components, positioned next to each other in a stacked arrangement. Each of the branches 935a and 935b can have an eyelet 934a and 934b that can accommodate the flexible member. In a stacked conformation, the eyelet 934a and 934b can be stacked together, so that the flexible member can pass through both openings 934a, 934b together in each loop. As shown in FIG. 19, the flexible member passes through the distal button 950 and both 934a and 934b in each loop. The proximal clamp 930 does not need to flex (or elastically or plastically deform) to allow the jaws to come together and close the clamping area.

FIG. 20 is a side view of the stabilization system of FIG. 19 without a proximal base, shown with distal mating surfaces of the clamp engaged with another mating surface, according to an illustrative embodiment. Turning to FIGS. 18-20, in various embodiments, the proximal button assembly 905 can be free of a proximal base. The protuberances 937a and 937b of the branches 935a and 935b can have clamping mating surfaces 942a and 942b. Clamping mating surfaces 942a and 942b can press against a proximal counterforce surface 944 such as a bone or other tissue, fixation plate, screw, suture anchor, nail, staple, bone graft, or other provider of resistance force or reaction force or Newtonian counterforce that provides a resistance or reaction force or bracing action to the clamping mating surface 942a, 942b. As the flexible member 170 is pulled in the proximal (tightening) direction, and the proximal button assembly 905 is pulled against the proximal counterforce surface 944. The clamping mating surfaces 942a, 942b can be at an oblique angle with respect to one another when the clamp is in a neutral state with the branches 935a, 935b in a relaxed state that can be generally parallel to one another and to the proximal-distal central axis of the device. In the neutral state, the clamping mating surfaces 942a, 942b slope down and out to form force receiving areas 943a, 943b that can be referred to as distal contact points or lower corners as shown in FIG. 20. The portions of the mating surfaces 942a, 942b that extend the furthest in the distal direction to form the force receiving areas 943a, 943b are also laterally distant from the proximal-distal central axis. As the flexible member is pulled tight and the clamping mating surfaces 942a, 942b are pulled against the proximal counterforce surface, the two separate stacked branches 935a and 935b can slightly rotate relative to each other about the stacked openings 934a and 934b, or more generally about some common pivoting point or points, as the jaws come together.

The axis of rotation that the stacked branches rotate about relative to each other can be parallel to, and below, the proximal counterforce surface, and a clamping plane, that can also be the coronal plane, that is perpendicular to the axis of rotation of the stacked branches 935a and 935b can also be perpendicular to the proximal counterforce surface and parallel to the distal-to-axial central axis of the clamp. The portions of the protuberances 937a, 937b that are arranged along the clamping plane can be the portions of the protuberances 937a, 937b that extend the furthest in the distal direction relative to portions of the protuberances in other radial locations. Force receiving areas 943a, 943b can be the most radially distant portions of the protuberances along the clamping plane, and force receiving areas 943a, 943b can be the portions of the protuberances that extend the furthest in the distal direction.

As the flexible member is pulled tight in the proximal direction, the force receiving areas 943a, 943b can engage with the proximal counterforce surface 944 first. As additional pressure is applied by pulling the ends of the flexible member in the proximal direction, the protuberances can rock towards each other around the points where the force receiving areas are engaged with the proximal counterforce surface. As additional pressure is applied by pulling the ends of the flexible member in the proximal direction, clamping force is generated by the proximal counterforce surface 944 pressing against the force receiving areas 943a, 943b, which can also be lower corners, and rocking the protuberances towards each other. As force is applied to the lower corners at the force receiving areas 943a, 943b, the jaws can be pushed together and the clamping area can be closed around the flexible member.

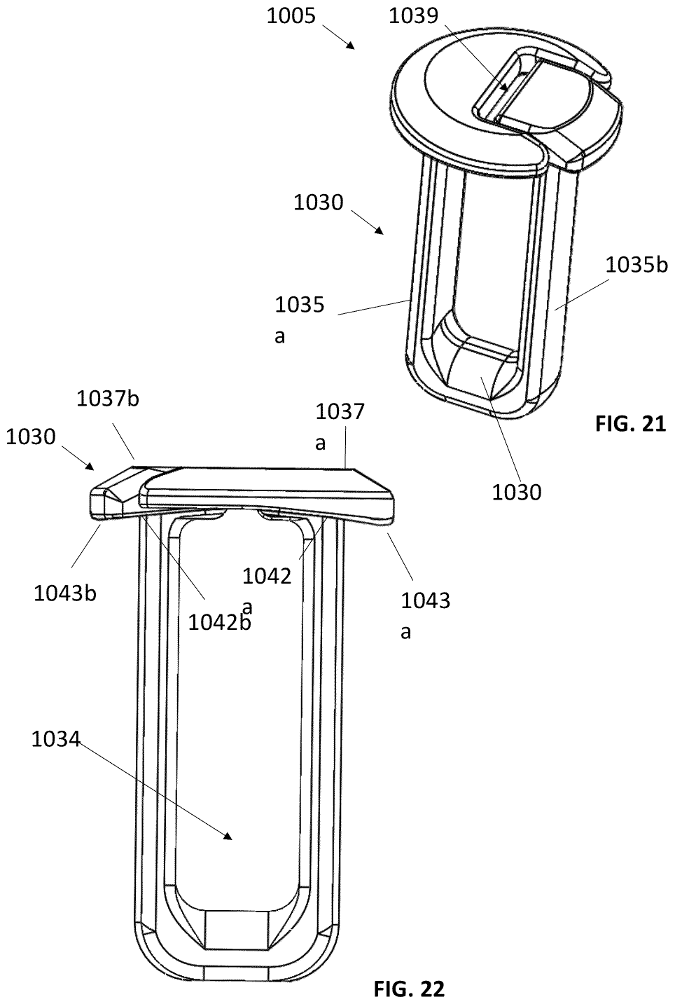

FIG. 21 is a perspective view of a one-piece proximal button assembly without a proximal base, according to an illustrative embodiment, and FIG. 22 is a side view of the one-piece proximal button assembly without a proximal base of FIG. 21, according to the illustrative embodiment. The one-piece proximal button assembly 1005 can have a saddle 1033 and branches 1035, with an opening 1034 above the saddle and between the branches. The branches 1035 can have protuberances 1037, and protuberances 1037 can have force receiving areas 1043. A clamping space 1039 can be between the protuberances 1037.

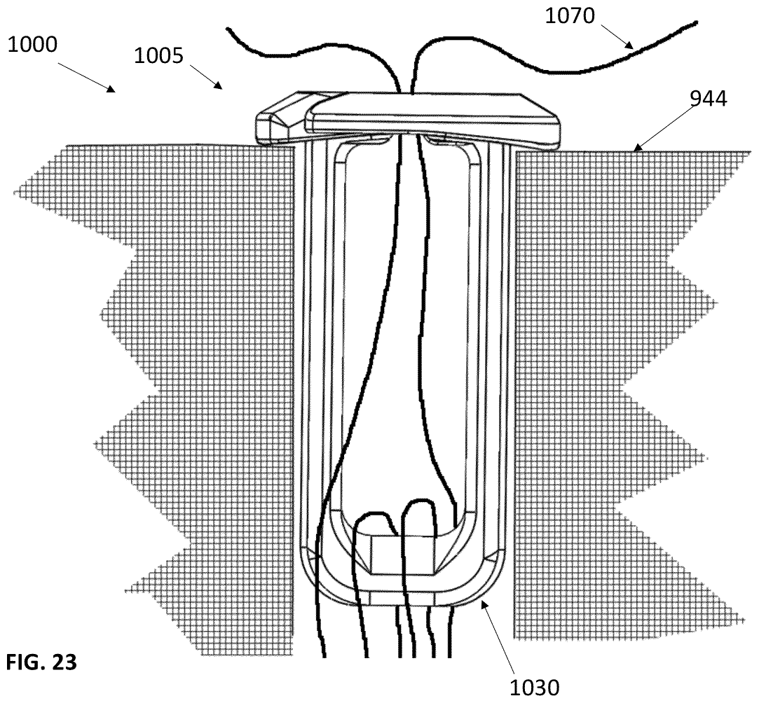

FIG. 23 is a side view of a stabilization system 1000 utilizing the one-piece clamp 1030 without a proximal base of FIG. 21, according to an illustrative embodiment. Turning to FIGS. 21-23, in various embodiments, the one-piece proximal button assembly 1005 can be free of a proximal base. The protuberances 1037a and 1037b of the branches 1035a and 1035b can have clamping mating surfaces 1042a and 1042b. Clamping mating surfaces 1042a and 1042b can press against a proximal counterforce surface 944 such as a bone or other tissue, fixation plate, screw, suture anchor, nail, staple, bone graft, or other provider of Newtonian counterforce that provides a Newtonian counterforce to the clamping mating surface 1042a, 1042b. As the flexible member 170 is pulled in the proximal (tightening) direction, and the proximal button assembly 1005 is pulled against the proximal counterforce surface 944. The clamping mating surfaces 1042a, 1042b can be at an oblique angle with respect to one another when the clamp is in a neutral state with the branches 1035a, 1035b in a relaxed, non-flexed state that can be generally parallel to one another and to the proximal-distal central axis of the device. In the neutral state, the clamping mating surfaces 1042a, 1042b slope down and out to form force receiving areas 1043a, 1043b that can be referred to as distal contact points or lower corners as shown in FIG. 22. The portions of mating surfaces 1042a, 1042b that extend the furthest in the distal direction to form the force receiving areas 1043a, 1043b are also laterally distant from the proximal-distal central axis. As the flexible member is pulled tight and the clamping mating surfaces 1042a, 1042b are pulled against the proximal counterforce or mating surface, the two branches 1035a and 1035b can flex towards each other along a clamping plane as the jaws come together.

The portions of the protuberances 1037a, 1037b that are arranged along the clamping plane can be the portions of the protuberances 1037a, 1037b that extend the furthest in the distal direction relative to portions of the protuberances in other radial locations. Force receiving areas 1043a, 1043b can be lower corners that are the most radially distant portions of the protuberances along the clamping plane, and the lower corners of the force receiving areas 1043a, 1043b can be the portions of the protuberances that extend the furthest in the distal direction.

As the flexible member is pulled tight in the proximal direction, the force receiving areas 1043a, 1043b can engage with the proximal counterforce surface 944 first. As additional pressure is applied by pulling the ends of the flexible member in the proximal direction, the protuberances can rock towards each other around the points where the force receiving areas are engaged with the proximal counterforce surface. As additional pressure is applied by pulling the ends of the flexible member in the proximal direction, clamping force is generated by the proximal counterforce surface 944 pressing against the force receiving areas 1043a, 1043b and rocking the protuberances towards each other. As force is applied to the force receiving areas 1043a, 1043b, the jaws can be pushed together and the clamping area can be closed around the flexible member. As the flexible member is pulled tight, the stabilization system can tighten on the anatomical structure to stabilize various anatomical structures, and the jaws can close around the flexible member to lock the stabilization system in place and make it resist elongation (or lengthening or loosening).

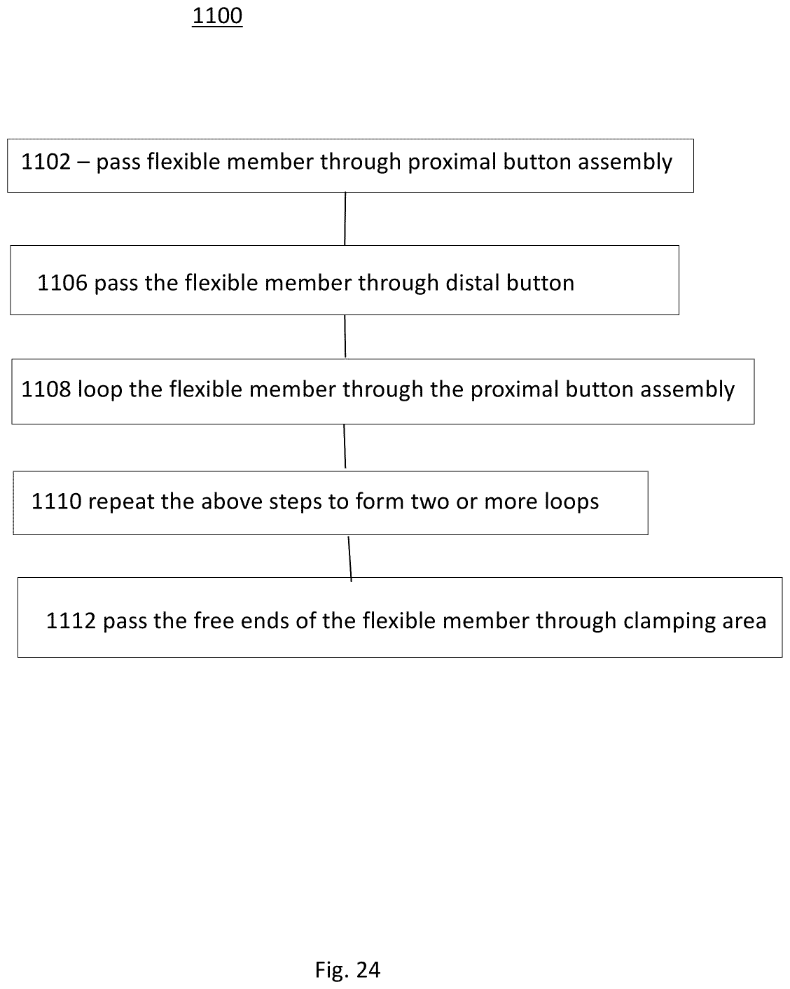

FIG. 24 is a diagram of a method 1100 of assembling a stabilization system that can be used to stabilize an anatomical structure during a surgery without tying a knot (free from knots), according to an illustrative embodiment. At box 1102, a flexible member can be passed though an opening in a proximal button assembly. At optional box 1106, the flexible member can be passed through an opening in a distal button. At box 1108, the flexible member can be routed through the opening in the proximal button assembly to form a loop in the flexible member. At box 1110, the above steps can be repeated to form two or more loops in the flexible member. At box 1112, the free ends of the flexible member can be passed through a clamping area of the proximal button assembly.

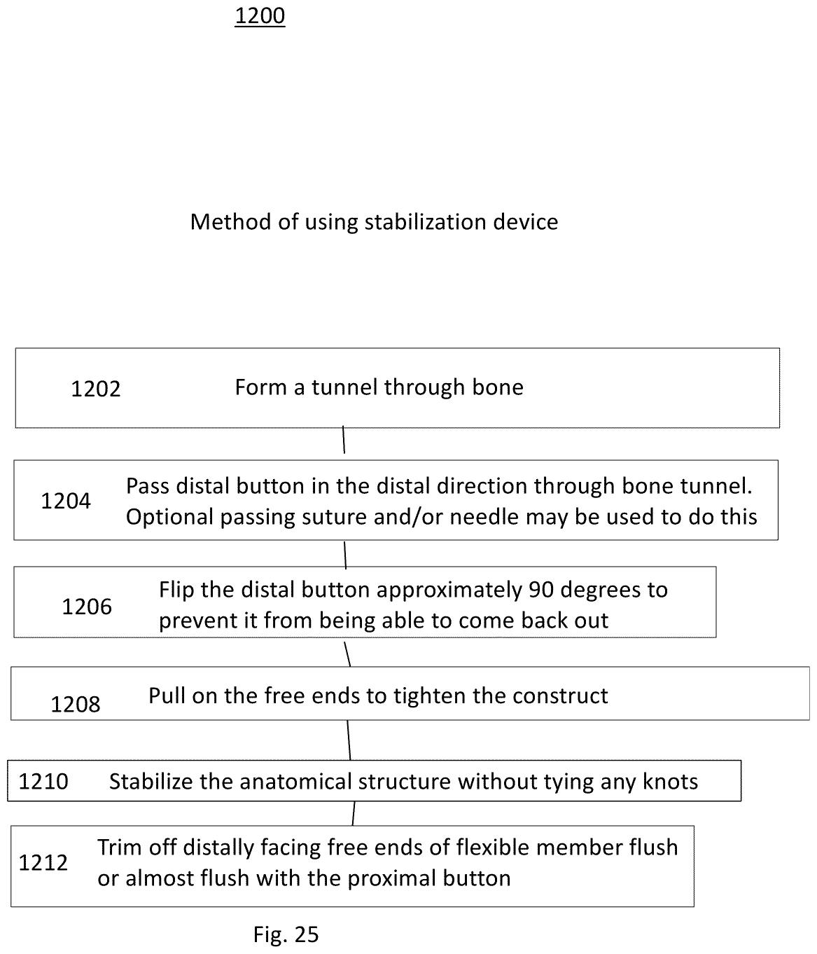

FIG. 25 is a diagram of a method 1200 of using a stabilization device to stabilize an anatomical structure during surgery, according to an illustrative embodiment. At box 1202, a tunnel can be formed through a bone. At box 1204, a distal button can be passed in the distal direction through the tunnel in the bone. In various embodiments, an optional passing suture and/or needle can be used to assist in passing the distal button through the tunnel in the bone. At box 1206, the distal button can be flipped 90 degrees after it has been pulled through the bone tunnel. This can prevent the distal button from being pulled back through the bone tunnel in the proximal direction. At box 1208, the free ends of the flexible member can be pulled to tighten the stabilization system. At box 1210, the anatomical structure can be stabilized without any knots (free of knots) because the clamp is holding the flexible member in place, which is in turn keeping the clamping pressure on the anatomical structure without the need for knots At box 1212 the excess free ends of the flexible member can be trimmed off on the proximal side of the proximal button. The free ends can be trimmed off flush or almost flush with the proximal button, and with no knots in the system.

In various embodiments, the flexible member can be manufactured out of any suitable material, including but not limited to polymers (e.g. UHMWPE, Polyester, Nylon, PEEK and PEKK among others), and metals or metal alloys (e.g. wire rope or chain or filament made out of Titanium, Cobalt, Molybdenum, Rhenium, Nickel, Iron or their alloys among others). Various other components may be manufactured out of any suitable material, including metals or metal alloys (e.g. Titanium, Cobalt, Molybdenum, Rhenium, Nickel, Iron, Aluminum or their alloys among others), ceramics or ceramic composites or suitable polymers or polymer composites (e.g. PEEK, PEK, PEKK, UHMWPE or carbon fibre- or other fibre-reinforced varieties of the above among others).

The words used in this specification to describe the embodiments herein are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are also meant to include not only the combination of elements which are literally set forth, but all equivalent structure, material or acts for performing substantially the same function in substantially the same way to obtain substantially the same result. The description included herein should not be taken as a limitation on the scope of the present invention or method of use.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, in various embodiments, multiple distal buttons can be used to form various loops such as a triangular loop or other shapes through and/or around various anatomical structures.

Also, as used herein, various directional and orientational terms (and grammatical variations thereof) such as “vertical”, “horizontal”, “up”, “down”, “bottom”, “top”, “side”, “front”, “rear”, “left”, “right”, “forward”, “rearward”, and the like, are used only as relative conventions and not as absolute orientations with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Accordingly, this description is meant to be taken only by way of example, and not to otherwise limit the scope of this invention.

What is claimed is:

1. A fixation system for knotlessly securing a flexible member stabilizing an anatomical structure, the system comprising:
a clamp comprising:
a first branch and a second branch, the first branch and the second branch adapted to move towards each other to close the clamp;
one or more openings at a distal end of at least one of the first or the second branch, the openings adapted for a flexible member to be passed through them to form two or more loops around the one or more saddles and a distal button;
a first protuberance at a proximal end of the first branch;
a second protuberance at a proximal end of the second branch; and
a first jaw on the first protuberance and a second jaw on the second protuberance, the first and second jaws defining a clamping area, the first and second jaws adapted for clamping and knotlessly securing the flexible member within the clamping area between the jaws.

2. The fixation system of claim 1, wherein the clamp further comprises a force receiving area on each of the protuberances, the force receiving areas laterally distant from a distal-proximal central axis of the clamp, the force receiving areas adapted to receive contact force, thereby urging the first branch and the second branch towards each other.

3. The fixation system of claim 2, further comprising a proximal base having a pocket configured to accommodate the protuberances, the pocket having an opening to allow the one or more saddles and the branches to extend through the opening in a distal direction.

4. The fixation system of claim 3, wherein the proximal base further comprises a sloped mating ramp on an interior of the pocket, the sloped mating ramp configured to engage with the force receiving areas and apply contact force to the force receiving areas, thereby urging the first branch and the second branch towards each other, when a force is applied to the one or more saddles in a distal direction.

5. The fixation system of claim 4, further comprising an expanded footprint washer, the expanded footprint washer having a washer recess configured to accommodate the proximal base, the washer recess having an opening to allow the one or more saddles and the branches to extend through the opening in a distal direction, the washer recess having a distal surface adapted for contact with an anatomical structure.

6. The fixation system of claim 2, wherein the force receiving areas are a distalmost portion of a distal surface of the protuberances.

7. The fixation system of claim 2, wherein one or more saddles is a single saddle, and the first branch and the second branch extend proximally from the saddle, the first branch and the second branch flexing slightly towards each other at their proximal ends to bring the jaws together when a force is applied to the saddle in a distal direction.

8. The fixation system of claim 2, wherein the one or more saddles is a first saddle and a second saddle, the first saddle on a proximal portion of the first branch and the second saddle on a proximal portion of the second branch, the first saddle and the second saddle configured to be arranged laterally distant from each other so that the flexible member can be looped through each saddle separately, wherein the first protuberance and the second protuberance are urged towards each other when a force is applied to the first saddle and the second saddle in a distal direction.

9. The fixation system of claim 2, wherein the one or more saddles is a first saddle and a second saddle, the first saddle on a proximal portion of the first branch and the second saddle on a proximal portion of the second branch, the first saddle and the second saddle configured to be stacked together so that the flexible member can be looped through both saddles together, wherein the first protuberance and the second protuberance are urged towards each other when a force is applied to the first saddle and the second saddle in a distal direction.

10. The fixation system of claim 3, wherein at least one of the branches has a barb extending out from the at least one branch, the barb adapted to engage with a mating lip of the proximal base to prevent the clamp from moving in a proximal direction relative to the proximal base after the barb is engaged with the mating lip.

11. A fixation system for knotlessly stabilizing anatomical structures, the system comprising:
a flexible member; and
a proximal button assembly, the proximal button assembly comprising:
one or more saddles, the one or more saddles adapted for the flexible member to be wrapped around the one or more saddles to form two or more loops around the one or more saddles and an anatomical structure of a patent;
a first protuberance having a first jaw, and a second protuberance having a second jaw, the first jaw and the second jaw defining a clamping area, wherein the flexible member is adapted to be passed through an anatomical structure of a patient to form a loop through the anatomical structure, and wherein the flexible member is adapted to be passed through the clamping area of the proximal button assembly, the clamping area adapted to clamp onto the flexible member and lock the flexible member in the clamping area;
the one or more saddles operatively connected to the first protuberance and the second protuberance, whereby a force applied in a distal direction to the one or more saddles by the flexible member causes the first protuberance and the second protuberance to be urged together, thereby closing the clamping area around the flexible member.

12. The fixation system of claim 11, further comprising a proximal base having a pocket configured to accommodate the protuberances, the pocket having an opening to allow the one or more saddles and the flexible member to extend through the opening in a distal direction.

13. The fixation system of claim 12, wherein the proximal base further comprises a sloped mating ramp on an interior of the pocket, the sloped mating ramp configured to engage with the protuberances and urge the protuberances towards each other when a force is applied to the one or more saddles in a distal direction.

14. The fixation system of claim 11, further comprising a distal button having a pair of openings adapted for the flexible member to be routed through the distal button as part of the two or more loops around the anatomical structure.

15. A method of stabilizing anatomical structures without the need for knots comprising:
providing a flexible member;
providing a proximal button assembly comprising:
one or more saddles, the one or more saddles adapted for the flexible member to be wrapped around the one or more saddles;
a first protuberance having a first jaw, and a second protuberance having a second jaw, the first jaw and the second jaw defining a clamping area adapted to clamp onto a flexible member and lock the flexible member in the clamping area without the need for tying knots;
the one or more saddles operatively connected to the first protuberance and the second protuberance, whereby a force applied in a distal direction to the one or more saddles by the flexible member causes the first protuberance and the second protuberance to be urged together, thereby closing the clamping area around the flexible member;
passing the flexible member through an anatomical structure of a patient to form at least two loops through the anatomical structure and the one or more saddles;
pulling on the free ends of the flexible member through the clamping area between the first jaw and the second jaw in a proximal direction to tighten the loops around the anatomical structure;
pulling on the free ends of the flexible member so that the tightening loops apply distally-directed force to the saddles, thereby urging the first and second protuberances together and thereby clamping the jaws together to lock the flexible member in place; and
releasing the tension on the free ends of the flexible member, resulting in the proximal button assembly locking the flexible member in place without the need for tying knots.
